(19) United States
(12) Patent Application Publication
(10) Pub. No.: US 2022/0226534 A1
(43) Pub. Date: Jul. 21, 2022

(54) MANUFACTURING METHOD OF HIGH-EFFICIENCY PLANT COMPOSITE TYPE BACTERIOSTATIC DEODORANT BASED ON HQ22 BEAN EXTRACT

(71) Applicant: NINGBO RHYSUAIR BIOTECHNOLOGY CO.,LTD., Ningbo City (CN)

(72) Inventor: Jian ZHOU, Ningbo City (CN)

(21) Appl. No.: 17/160,181

(22) Filed: Jan. 27, 2021

(30) Foreign Application Priority Data
Jan. 15, 2021 (CN) 2021-10052160.0

(51) Int. Cl. A61L 11/00; C12P 1/04

(52) U.S. Cl. CPC A61L 11/00; C12P 1/04

(57) ABSTRACT

The manufacturing method of high-efficiency plant composite type bacteriostatic deodorant based on HQ22 bean extract includes pretreating the leaves of certain beans and certain plants in the early stage, which are then respectively processed into powder by a pulverizer; uniformly mixing the two powders according to a certain mass ratio, which are then treated with inoculation process with a certain inoculation amount of Bacillus natto seed solution; carrying out fermentation treatment on that mixture to obtain dark brown fermentation liquor; separating and purifying the fermentation liquor to obtain active components of the high-efficiency bacteriostatic deodorant; mixing the active ingredient with a certain proportion of surfactant, buffer, solvent and flavoring agent; and then obtaining high-efficiency plant composite type bacteriostatic deodorant based on HQ22 bean extract was prepared. The method has the advantage of high-efficiency of deodorization and bacteriostasis.

### CROSS-REFERENCE TO RELATED APPLICATIONS

See Application Data Sheet.

### STATEMENT REGARDING FEDERALLY SPONSORED RESEARCH OR DEVELOPMENT

Not applicable.

### THE NAMES OF PARTIES TO A JOINT RESEARCH AGREEMENT

Not applicable.

### INCORPORATION-BY-REFERENCE OF MATERIAL SUBMITTED ON A COMPACT DISC OR AS A TEXT FILE VIA THE OFFICE ELECTRONIC FILING SYSTEM (EFS-WEB)

Not applicable.

### STATEMENT REGARDING PRIOR DISCLOSURES BY THE INVENTOR OR A JOINT INVENTOR

Not applicable.

### BACKGROUND OF THE INVENTION

### 1. Field of the Invention

The present invention belongs to the fields of family safety, harmlessness, environmental protection, bacteriostasis and deodorization, and specifically to a manufacturing method of plant composite type efficient bacteriostatic deodorant based on HQ22 soybean extract.

### 2. Description of Related Art Including Information Disclosed Under 37 CFR 1.97 and 37 CFR 1.98

In recent years, with the development of biotechnology, people's requirements for the quality of living environment are becoming more and more stringent. During the indoor storage of domestic garbage, a large amount of gas with pungent odor will be produced, which will not only pollute the surrounding air environment, but also adversely affect the health of residents. In developed countries such as Japan, biological deodorization is widely used in many families. The principle of biological deodorization is to use the efficient decomposition ability of microorganisms to decompose smelly harmful substances into non-toxic and harmless substances. Compared with the most popular fragrance masking method in China, biological deodorization is obviously more scientific, safe and environmentally friendly.

However, due to market reasons, some biological deodorization products, which are in pursuit of effectiveness, are mostly added with a large number of harmful chemical components to promote the decomposition efficiency of microorganisms. unfortunately, chemical component residues can easily pollute indoor air, and such components have no peculiar smell but are harmful to human body after long-term contact, so it is difficult to detect them in ordinary families without professional detection tools.

Therefore, a safe, harmless, and environment-friendly, high-efficiency plant composite biological bacteriostatic deodorant which can not only phagocytize and decompose odor and bacteria, but also inhibit the reproduction of surrounding bacteria will have a good application prospect in the field of bacteriostatic deodorization.

### BRIEF SUMMARY OF THE INVENTION

In view of the shortcomings of the existing biological bacteriostatic and deodorizing technology, the present invention takes safe and harmless HQ22 bean extracts (natto bacteria, nattokinase, soybean amino acids) and plant leaves (polyphenols) as raw materials, uses a plant composite biological degerming agent that is finally formed through certain treatment, as an effective component, and forms through a simple, safe and environmentally-friendly preparation process, an efficient bacteriostatic deodorant which can not only swallow and decompose odor and bacteria, but also inhibit the reproduction of surrounding bacteria.

The present invention relates to manufacturing method of high-efficiency plant composite type bacteriostatic deodorant based on HQ22 bean extract, which mainly comprises the following steps:
(1) selecting and cleaning some plump, mildew-free beans and fresh plant leaves, deactivating them at 60° C., drying them, and then respectively treating them with a pulverizer to obtain powder for later use;
(2) uniformly mixing the two powders according to a certain mass ratio, and mixing and inoculating the two powders with a certain amount of Bacillus natto seed solution according to a certain proportion;
(3) carrying out liquid fermentation treatment on the mixture to obtain dark brown fermentation liquor;
(4) separating and purifying the fermentation liquor to obtain the effective components of the high-efficiency bacteriostatic deodorant;
(5) mixing the effective components with a certain proportion of surfactant, buffer, solvent and flavoring agent to prepare the plant composite high-efficiency antibacterial deodorant based on the HQ22 bean extract.

The manufacturing method of high-efficiency plant composite type bacteriostatic deodorant based on HQ22 bean extract is characterized in that: in the step (1), the beans refer to one or more of Hokkaido soybeans, organically cultivated soybeans produced in Kyushu, soybeans produced in Okinawa, black soya beans, mung beans, peeled mung beans, red beans and kidney beans; and the beans is preferably one or two of Japanese Hokkaido soybean, organically cultivated soybean in Kyushu, soybean in Okinawa and mung bean with faded skin.

The manufacturing method of high-efficiency plant composite type bacteriostatic deodorant based on HQ22 bean extract is characterized in that: in the step (1), the plant leaves refer to one or more of persimmon leaf, tea, cypress leaf, heather leaves and wintergreen; preferably one or more of heather leaves, wintergreen and tea leaves.

The manufacturing method of high-efficiency plant composite type bacteriostatic deodorant based on HQ22 bean extract is characterized in that: in the step (1), the cleaning means washing with water for 2-10 times, preferably 3˜5 times.

The manufacturing method of high-efficiency plant composite type bacteriostatic deodorant based on HQ22 bean extract is characterized in that: in the step (1), the stewing means high pressure or normal pressure stewing at 80˜160° C. for 20 min˜1 hour, preferably high pressure stewing at 90˜120° C. for 30˜45 min.

The manufacturing method of high-efficiency plant composite type bacteriostatic deodorant based on HQ22 bean extract is characterized in that: in the step (1), the drying means drying at 40-160° C. for 2-20 hours, preferably drying at 80˜120° C. for 8˜16 hours.

The manufacturing method of high-efficiency plant composite type bacteriostatic deodorant based on HQ22 bean extract is characterized in that: in the step (1), the yellow powder is filtered by a 50-2000 mesh filter screen, and preferably a 1000-1800 mesh filter screen.

The manufacturing method of high-efficiency plant composite type bacteriostatic deodorant based on HQ22 bean extract is characterized in that: in the step (2), the certain mass ratio means that the mass ratio of the bean powder and the plant leaf powder is 1: 9-9:1, preferably 9:1˜7:3.

The manufacturing method of high-efficiency plant composite type bacteriostatic deodorant based on HQ22 bean extract is characterized in that: in the step (2), the Bacillus natto seed solution means that the inoculated bacterial powder is placed in a sterile liquid culture medium and cultured at a constant temperature of 10-45° C. for 8-20 hours, and the pH value of the culture medium is 6.0-12.0; preferably the temperature is 25˜45° C., the time is 18˜24 hours, and pH value is 6.0˜8.0.

The manufacturing method of high-efficiency plant composite type bacteriostatic deodorant based on HQ22 bean extract is characterized in that: in the step (2), the certain proportion refers to 1%˜45% (in terms of powder mass), preferably 10˜30%.

The manufacturing method of high-efficiency plant composite type bacteriostatic deodorant based on HQ22 bean extract is characterized in that: in the step (2), the inoculation amount is 1˜15%, preferably 3˜8%.

The manufacturing method of high-efficiency plant composite type bacteriostatic deodorant based on HQ22 bean extract is characterized in that: in the step (3), in the liquid fermentation treatment, the fermentation temperature is 20˜50° C., the water addition amount is 1%˜50%, and the fermentation time is 2˜10 days; preferably the temperature is 20˜40° C., the water addition amount is 20%˜50%, and the time is 5˜8 days.

The manufacturing method of high-efficiency plant composite type bacteriostatic deodorant based on HQ22 bean extract is characterized in that: in the step (4), the separation and purification refer to one or more of column chromatography, ultrafiltration, extraction, affinity particle and integrated separation and purification, and are performed at 20-50° C.; the separation and purification refer to preferably ultrafiltration and/or integrated separation and purification.

The manufacturing method of high-efficiency plant composite type bacteriostatic deodorant based on HQ22 bean extract is characterized in that: in the step (5), the certain proportion refers to 1-20% of active components, 0.05-5% of surfactant, 0.1-2.0% of buffer, 50-99% of solvent and 0.5-2.0% of flavoring agent; preferably 5˜15% of active components, 0.5˜3% of surfactant, 0.5˜1.5% of buffer, 70˜99% of solvent and 1˜1.5% of flavoring agent.

The manufacturing method of high-efficiency plant composite type bacteriostatic deodorant based on HQ22 bean extract is characterized in that: in the step (5), the surfactant refers to one or two of dodecyl amino dipropionate amphoteric surfactant and didecyldimethylammonium chloride cationic surfactant; and the surfactant preferably refers to dodecyl dipropionate amphoteric surfactant.

The manufacturing method of high-efficiency plant composite type bacteriostatic deodorant based on HQ22 bean extract is characterized in that: in the step (5), the buffering agent refers to one or more of citrate, ethylenediamine tetraacetate and acetate; preferably refers to One or two of citrate and acetate.

The manufacturing method of high-efficiency plant composite type bacteriostatic deodorant based on HQ22 bean extract is characterized in that: in the step (5), the flavoring agent refers to plant-type natural fruit fragrance, including citrus extract, lemon extract, clove extract, etc.

The manufacturing method of high-efficiency plant composite type bacteriostatic deodorant based on HQ22 bean extract is characterized in that: in the step (5), the solvent is one or more of propylene glycol, ethanol and deionized water; and one or more of propylene glycol and deionized water.

The present invention takes safe and harmless HQ22 bean extracts (natto bacteria, nattokinase, soybean amino acids) and plant leaves (polyphenols) as raw materials, and finally forms a plant composite biological degerming agent as an active component, which uses natto bacteria and nattokinase to swallow and decompose odors and bacteria, and at the same time, uses the interface impedance of soybean amino acids and polyphenols formed by them to inhibit surrounding bacteria. The method has the advantage of high-efficiency of deodorization and bacteriostasis, so it has a good application prospect in the fields of safety, harmlessness, environmental protection, bacteriostasis and deodorization.

### BRIEF DESCRIPTION OF THE SEVERAL VIEWS OF THE DRAWINGS

FIG. 1 is a graph illustrating the bacteriostatic effect of different kinds of beans on Flavobacterium in Embodiments 1-3 of the present invention.

FIG. 2 is a graph illustrating bacteriostatic effect of different kinds of beans on Enterobacter in Embodiments 1-3 of the present invention.

FIG. 3 is a graph illustrating bacteriostatic effect of different kinds of beans on mould in Embodiments 1-3 of the present invention.

FIG. 4 is a graph illustrating ammonia odor removal effect with different contents of active components manufactured in Embodiments 3-5 of the present invention.

FIG. 5 is a graph illustrating hydrogen sulfide odor removal effect with different contents of active components manufactured in Embodiments 3-5 of the present invention.

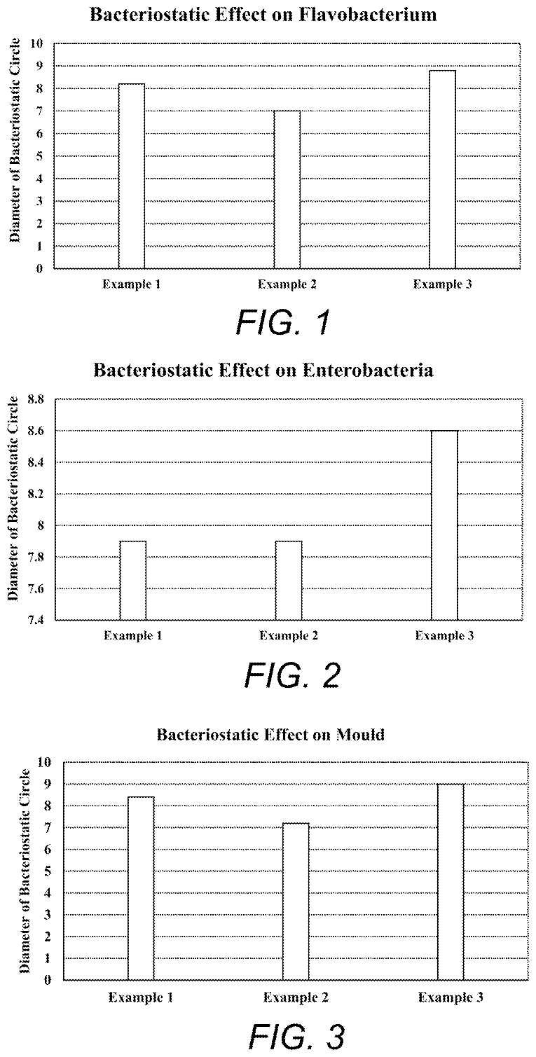

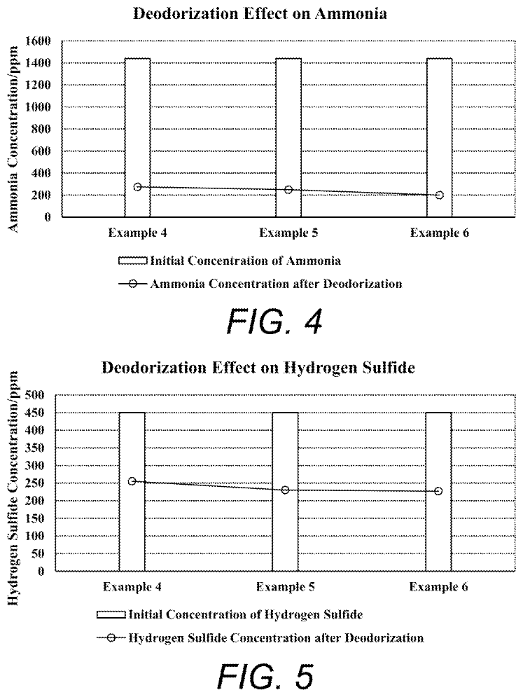

### DETAILED DESCRIPTION OF THE INVENTION

The method of the present invention will be described in detail with specific examples, which are implemented on the premise of the technical solutions of the present invention, but the protection scope of the present invention is not limited to the following examples.

### Embodiment 1

The present embodiment relates to a manufacturing method of high-efficiency plant composite type bacteriostatic deodorant based on HQ22 bean extract. Some plump and mildew-free Japanese Hokkaido soybeans and fresh photinia leaves were washed with deionized water for 3˜5 times, stewed at 120° C. for 45 min under high pressure, and dried at 120° C. for 16 hours. The above soybeans and photinia leaves are decomposed with a pulverizer, thus pulverizing them to obtain powder, which is filtered with a 1500 mesh filter screen for later use. The inoculated bacterial powder was placed in a sterile liquid medium with pH value of 6.8 and was cultured at 45° C. for 24 hours to obtain Bacillus natto seed solution. Japanese Hokkaido soybean powder and photinia leave powder are uniformly mixed according to a ratio of 9:1 and sterilized. Then, the mixed powder with the mass ratio of 25% and the Bacillus natto seed solution with the inoculation amount of 5% are mixed and inoculated; after that, at the temperature of 40° C. and the water content of 50%, it was subjected to liquid fermentation for 7 days to obtain dark brown fermentation liquor. The fermentation liquor was centrifuged at 10000 r/m in for 10 min and then was sterilized and treated by reverse osmosis membrane; and then the filtrate was passed through ultrafiltration membrane with cutoff molecular weight of 8 ku to remove impurities with small molecular weight, and then eluted with agar gel column to obtain active substance. The above filtrate was concentrated by ultrafiltration equipment, then filtered by gel column, and centrifuged at 12000 r/min for 10 min to obtain high purity active components. According to a mass ratio of 7% of active ingredients, 0.5% of dodecyl amino dipropionate amphoteric surfactant, 1.5% of citrate, 90% of propylene glycol, 1% of deionized water and 1% of citrus extract, they are mixed and stirred evenly, thus obtaining the plant composite type high-efficiency bacteriostatic deodorant based on HQ22 bean extract.

### Embodiment 2

The present embodiment relates to a manufacturing method of high-efficiency plant composite type bacteriostatic deodorant based on HQ22 bean extract. Some plump and mildew-free black soya beans and fresh photinia leaves were washed with deionized water for 3˜5 times, stewed at 120° C. for 45 min under high pressure, and dried at 120° C. for 16 hours. The above black soya beans and photinia leaves are decomposed with a pulverizer, thus pulverizing them to obtain powder, which is filtered with a 1500 mesh filter screen for later use. The inoculated bacterial powder was placed in a sterile liquid medium with pH value of 6.8 and was cultured at 45° C. for 24 hours to obtain Bacillus natto seed solution. Black soya bean powder and photinia leave powder are uniformly mixed according to a ratio of 9:1 and sterilized. Then, the mixed powder with the mass ratio of 25% and the Bacillus natto seed solution with the inoculation amount of 5% are mixed and inoculated; after that, at the temperature of 40° C. and the water content of 50%, it was subjected to liquid fermentation for 7 days to obtain dark brown fermentation liquor. The fermentation liquor was centrifuged at 10000 r/m in for 10 min and then was sterilized and treated by reverse osmosis membrane; and then the filtrate was passed through ultrafiltration membrane with cutoff molecular weight of 8 ku to remove impurities with small molecular weight, and then eluted with agar gel column to obtain active substance. The above filtrate was concentrated by ultrafiltration equipment, then filtered by gel column, and centrifuged at 12000 r/min for 10 min to obtain high purity active components. According to a mass ratio of 7% of active ingredients, 0.5% of dodecyl amino dipropionate amphoteric surfactant, 1.5% of citrate, 90% of propylene glycol, 1% of deionized water and 1% of citrus extract, they are mixed and stirred evenly, thus obtaining the plant composite type high-efficiency bacteriostatic deodorant based on HQ22 bean extract.

### Embodiment 3

The present embodiment relates to a manufacturing method of high-efficiency plant composite type bacteriostatic deodorant based on HQ22 bean extract. Some plump and mildew-free peeled mung beans and fresh photinia leaves were washed with deionized water for 3˜5 times, stewed at 120° C. for 45 min under high pressure, and dried at 120° C. for 16 hours. The above peeled mung beans and photinia leaves are decomposed with a pulverizer, thus pulverizing them to obtain powder, which is filtered with a 1500 mesh filter screen for later use. The inoculated bacterial powder was placed in a sterile liquid medium with pH value of 6.8 and was cultured at 45° C. for 24 hours to obtain Bacillus natto seed solution. The peeled mung bean powder and photinia leave powder are uniformly mixed according to a ratio of 9:1 and sterilized. Then, the mixed powder with the mass ratio of 25% and the Bacillus natto seed solution with the inoculation amount of 5% are mixed and inoculated; after that, at the temperature of 40° C. and the water content of 50%, it was subjected to liquid fermentation for 7 days to obtain dark brown fermentation liquor. The fermentation liquor was centrifuged at 10000 r/m in for 10 min and then was sterilized and treated by reverse osmosis membrane; and then the filtrate was passed through ultrafiltration membrane with cutoff molecular weight of 8 ku to remove impurities with small molecular weight, and then eluted with agar gel column to obtain active substance. The above filtrate was concentrated by ultrafiltration equipment, then filtered by gel column, and centrifuged at 12000 r/min for 10 min to obtain high purity active components. According to a mass ratio of 7% of active ingredients, 0.5% of dodecyl amino dipropionate amphoteric surfactant, 1.5% of citrate, 90% of propylene glycol, 1% of deionized water and 1% of citrus extract, they are mixed and stirred evenly, thus obtaining the plant composite type high-efficiency bacteriostatic deodorant based on HQ22 bean extract.

### Embodiment 4

The present embodiment relates to a manufacturing method of high-efficiency plant composite type bacteriostatic deodorant based on HQ22 bean extract. Some plump and mildew-free peeled mung beans and fresh photinia leaves were washed with deionized water for 3˜5 times, stewed at 120° C. for 45 min under high pressure, and dried at 120° C. for 16 hours. The above peeled mung beans and photinia leaves are decomposed with a pulverizer, thus pulverizing them to obtain powder, which is filtered with a 1500 mesh filter screen for later use. The inoculated bacterial powder was placed in a sterile liquid medium with pH value of 6.8 and was cultured at 45° C. for 24 hours to obtain Bacillus natto seed solution. The peeled mung bean powder and photinia leave powder are uniformly mixed according to a ratio of 9:1 and sterilized. Then, the mixed powder with the mass ratio of 25% and the Bacillus natto seed solution with the inoculation amount of 7% are mixed and inoculated; after that, at the temperature of 40° C. and the water content of 50%, it was subjected to liquid fermentation for 7 days to obtain dark brown fermentation liquor. The fermentation liquor was centrifuged at 10000 r/m in for 10 min and then was sterilized and treated by reverse osmosis membrane; and then the filtrate was passed through ultrafiltration membrane with cutoff molecular weight of 8 ku to remove impurities with small molecular weight, and then eluted with agar gel column to obtain active substance. The above filtrate was concentrated by ultrafiltration equipment, then filtered by gel column, and centrifuged at 12000 r/min for 10 min to obtain high purity active components. According to a mass ratio of 7% of active ingredients, 0.5% of dodecyl amino dipropionate amphoteric surfactant, 1.5% of citrate, 90% of propylene glycol, 1% of deionized water and 1% of citrus extract, they are mixed and stirred evenly, thus obtaining the plant composite type high-efficiency bacteriostatic deodorant based on HQ22 bean extract.

### Embodiment 5

The present embodiment relates to a manufacturing method of high-efficiency plant composite type bacteriostatic deodorant based on HQ22 bean extract. Some plump and mildew-free peeled mung beans and fresh photinia leaves were washed with deionized water for 3˜5 times, stewed at 120° C. for 45 min under high pressure, and dried at 120° C. for 16 hours. The above peeled mung beans and photinia leaves are decomposed with a pulverizer, thus pulverizing them to obtain powder, which is filtered with a 1500 mesh filter screen for later use. The inoculated bacterial powder was placed in a sterile liquid medium with pH value of 6.8 and was cultured at 45° C. for 24 hours to obtain Bacillus natto seed solution. The peeled mung bean powder and photinia leave powder are uniformly mixed according to a ratio of 9:1 and sterilized. Then, the mixed powder with the mass ratio of 25% and the Bacillus natto seed solution with the inoculation amount of 10% are mixed and inoculated; after that, at the temperature of 40° C. and the water content of 50%, it was subjected to liquid fermentation for 7 days to obtain dark brown fermentation liquor. The fermentation liquor was centrifuged at 10000 r/min for 10 min and then was sterilized and treated by reverse osmosis membrane; and then the filtrate was passed through ultrafiltration membrane with cutoff molecular weight of 8 ku to remove impurities with small molecular weight, and then eluted with agar gel column to obtain active substance. The above filtrate was concentrated by ultrafiltration equipment, then filtered by gel column, and centrifuged at 12000 r/min for 10 min to obtain high purity active components. According to a mass ratio of 7% of active ingredients, 0.5% of dodecyl amino dipropionate amphoteric surfactant, 1.5% of citrate, 90% of propylene glycol, 1% of deionized water and 1% of citrus extract, they are mixed and stirred evenly, thus obtaining the plant composite type high-efficiency bacteriostatic deodorant based on HQ22 bean extract.

Performance Tests

The test method of bacteriostatic effect in the implementation case is as follows: as the common microorganisms in municipal household garbage are Escherichia coli, Flavobacterium and mold, the bacteriostatic test takes these four strains as test objects; and the specific operation is as follows: 0.1 mL of bacteriostatic deodorant in the implementation case is coated on a flat plate; samples were put on filter paper, dried and put on the plate coated with indicator bacteria, and cultured for 24 h to measure the diameter of inhibition zone.

The test method of deodorization effect in the implementation case is as follows: as the common odor sources in municipal household garbage are ammonia and hydrogen sulfide, the deodorization test takes these two odor sources as test objects; and the specific operation is as follows: putting the bacteriostatic deodorant into a 50 ml sprayer and spray it into a 2500 ml experimental bottle; spraying 7 times, counting 1 ml; stirring to evenly distribute the gas, and detecting the odor concentration after 20 min.

1. A manufacturing method of high-efficiency plant composite type bacteriostatic deodorant based on HQ22 bean extract, the method comprising the following steps:
(1) selecting and cleaning some plump, mildew-free beans and fresh plant leaves, deactivating them at 60° C. drying them, and then respectively treating them with a pulverizer to obtain powder for later use;
(2) uniformly mixing the two powders according to a certain mass ratio, and mixing and inoculating the two powders with a certain inoculation amount of Bacillus natto seed solution according to a certain proportion;
(3) performing liquid fermentation treatment on the mixture obtained in step (2) to obtain dark brown fermentation liquor;
(4) separating and purifying the fermentation liquor obtained in step (3) to obtain active components of high-efficiency bacteriostatic deodorant; and
(5) mixing the active components with a certain proportion of surfactant, buffer, solvent and flavoring agent to obtain a plant composite type high-efficiency bacteriostatic deodorant based on the HQ22 bean extract.

2. The manufacturing method of high-efficiency plant composite type bacteriostatic deodorant based on HQ22 bean extract according to claim 1, wherein, in the step (1), the beans are comprised of one or more of Hokkaido soybeans, organically cultivated soybeans produced in Kyushu, soybeans produced in Okinawa, black soya beans, mung beans, peeled mung beans, red beans and kidney beans;
wherein the plant leaves are comprised of one or more of persimmon leaf, tea, cypress leaf, heather leaves and wintergreen; the cleaning means washing with water for 2-10 times; and
wherein the stewing comprises high pressure or normal pressure stewing at 80˜160° C. for 20 min˜1 hour; the drying means drying at 40-160° C. for 2-20 hours; the yellow powder is filtered by a 50-2000 mesh filter screen.

3. The manufacturing method of high-efficiency plant composite type bacteriostatic deodorant based on HQ22 bean extract according to claim 1, wherein, in the step (2), the certain mass ratio is comprised of the mass ratio of the bean powder and the plant leaf powder being between 1:9-9:1;
wherein the Bacillus natto seed solution is comprised of the inoculated bacterial powder i& placed in a sterile liquid culture medium and cultured at a constant temperature of 10-45° C. for 8-20 hours, and the pH value of the culture medium is 6.0-12.0;
wherein the certain proportion refers to 1%˜45% kin terms of powder mass; and
wherein the inoculation amount is 1-15%.

4. The manufacturing method of high-efficiency plant composite type bacteriostatic deodorant based on HQ22 bean extract according to claim 1, wherein, in the step (3), in the liquid fermentation treatment, the fermentation temperature is 20˜50° C., the water addition amount is 1%˜50%, and the fermentation time is 2˜10 days.

5. The manufacturing method of high-efficiency plant composite type bacteriostatic deodorant based on HQ22 bean extract according to claim 1, wherein, in the step (4), the separation and purification refer to one or more of column chromatography, ultrafiltration, extraction, affinity particle and integrated separation and purification, and are performed at 20-50° C.

6. The manufacturing method of high-efficiency plant composite type bacteriostatic deodorant based on HQ22 bean extract according to claim 1, wherein, in the step (5), the certain proportion is comprised of 1-20% of active components, 0.05-5% of surfactant, 0.1-2.0% of buffer, 50-99% of solvent and 0.5-2.0% of flavoring agent;
wherein the surfactant is comprised of one or two of dodecyl amino dipropionate amphoteric surfactant and didecyldimethylammonium chloride cationic surfactant;
wherein the buffering agent is comprised of one or more of citrate, ethylenediamine tetraacetate and acetate;
wherein the flavoring agent is comprised of plant-type natural fruit fragrance; and
wherein the solvent is comprised of one or more of propylene glycol, ethanol and deionized water.
